(19) United States
(12) Patent Application Publication
(10) Pub. No.: US 2007/0254357 A1
(43) Pub. Date: Nov. 1, 2007

(54) Gene therapy using replication competent targeted adenoviral vectors

(75) Inventors: Richard J. Gregory, Carlsbad, CA (US); Whei-Mei Huang, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Canji, Inc., San Diego, CA (US)

(21) Appl. No.: 11/818,844

(22) Filed: Jun. 15, 2007

Related U.S. Application Data
(63) Continuation of application No. 10/845,489, filed on May 12, 2004.
Application No. 10/845,489 is a continuation of application No. 09/215,644, filed on Dec. 16, 1998, now abandoned.
Application No. 09/215,644 is a continuation of application No. 08/433,798, filed on May 3, 1995, now abandoned.

(51) Int. Cl. C12N 15/00; A61P 35/00

(52) U.S. Cl. 435/320.1

(57) ABSTRACT

This invention provides a method of treating cancer by administering a replication competent adenoviral vector comprising a therapeutic gene and a disease specific gene regulatory region operationally linked to at least one replication gene. The replication competent targeted adenoviral vector preferentially replicates in the tumor cells following activation of the tumor specific gene regulatory region thereby amplifying the effect of the therapeutic gene carried by the replication competent adenoviral vector. This invention enables for the first time the targeting of a therapeutic gene for treating cancer using small amounts of viral vectors which selectively replicate to deliver therapeutic dosages of the therapeutic gene.

### BACKGROUND OF THE INVENTION

The present invention relates generally to gene therapy methods for the treatment of diseases and, more particularly cancer, through administration of a replication competent targeted virus comprising a therapeutic gene and a tumor specific enhancer/promoter upstream of an essential viral gene wherein the cancer cell activates the tumor specific promoter causing the virus to replicate thereby amplifying the cytotoxic effect of the therapeutic gene.

The goal of gene therapy in treating abnormal pathological conditions such as cancer is to reestablish the normal control of cellular proliferation or to eliminate the cells undergoing aberrant proliferation. There are three primary strategies by which in vivo genetic modification can lead to therapeutic benefit. These strategies include the enhancement of immunogenicity toward the aberrant cells, the correction of a genetic defect which leads to the aberrant phenotype and the delivery of a gene whose product is or can be made toxic to the recipient cells. Of all three strategies, the one most likely to provide the greatest benefit with the least side effects is to deliver the vector carrying the therapeutic gene to as many cells as possible while controlling the functional delivery of the therapeutic gene to the abnormally proliferating cells.

A specific example of correcting a genetic defect to reinstate control of normal cellular proliferation using, for example, p53 mediated gene therapy. p53 plays a central role in cell cycle progression, arresting growth so that repair or apoptosis can occur in response to DNA damage. Wild-type p53 has recently been identified as a necessary component for apoptosis induced by irradiation or treatment with some chemotherapeutic agents (Lowe et al. (1993) A and B). Due to the high prevalence of p53 mutations in human tumors, it is possible that tumors which have become refractory to chemotherapy and irradiation treatments may have become so due in part to the lack of wild-type p53. By providing functional p53, these tumors are susceptible to apoptosis normally associated with the DNA damage induced by radiation and chemotherapy.

As with treating p53 deficient tumors, gene therapy is equally applicable to other tumor suppressor genes which can be used either alone or in combination with therapeutic agents to control cell cycle progression of tumor cells and/or induce cell death. Moreover, genes which do not encode cell cycle regulatory proteins, but directly induce cell death such as suicide genes or, genes which are directly toxic to the cell can be used in gene therapy protocols to directly eliminate the cell cycle progression of tumor cells.

Regardless of which gene is used to reinstate the control of cell cycle progression, the rationale and practical applicability of this approach is identical. Namely, to achieve high efficiencies of gene transfer to express therapeutic quantities of the recombinant product. The choice of which vector to use to enable high efficiency gene transfer with minimal risk to the patient is therefore important to the level of success of the gene therapy treatment.

One of the critical points in successful gene therapy of cancer or certain other diseases is the ability to affect a significant fraction of the aberrant cells. The use of retroviral vectors has been largely explored for this purpose in a variety of tumor models. For example, in the treatment of hepatic malignancies, retroviral vectors have been employed with little success because these vectors are not able to achieve the high level of gene transfer required for in vivo gene therapy (Huber, B. E. et al., 1991; Caruso M. et al., 1993).

To achieve a more sustained source of virus production, researchers have attempted to overcome the problem associated with low level of gene transfer by direct injection of retroviral packaging cell lines into solid tumors (Caruso, M. et al., 1993; Ezzidine, Z. D. et al., 1991; Culver, K. W. et al., 1992). However, these methods are unsatisfactory for use in human patients because the method is troublesome and induces an inflammatory response against the packaging cell line in the patient. Another disadvantage of retroviral vectors is that they require dividing cells to efficiently integrate and express the recombinant gene of interest (Huber, B. E. 1991). Stable integration into an essential host gene can lead to the development or inheritance of pathogenic diseased states.

Recombinant adenoviruses have distinct advantages over retroviral and other gene delivery methods (for review, see Siegfried (1993)). Adenoviruses have never been shown to induce tumors in humans and have been safely used as live vaccines (Straus (1984)). Replication deficient recombinant adenoviruses can be produced by replacing the E1 region necessary for replication with the target gene. Adenovirus does not integrate into the human genome as a normal consequence of infection, thereby greatly reducing the risk of insertional mutagenesis possible with retrovirus or adeno-associated viral (AAV) vectors. This lack of stable integration also leads to an additional safety feature in that the transferred gene effect will be transient, as the extrachromosomal DNA will be gradually lost with continued division of normal cells. Stable, high titer recombinant adenovirus can be produced at levels not yet achievable with retrovirus or AAV, allowing enough material to be produced to treat a large patient population. Moreover, adenovirus vectors are capable of highly efficient in vivo gene transfer into a broad range of tissue and tumor cell types. For example, others have shown that adenovirus mediated gene delivery has a strong potential for gene therapy for diseases such as cystic fibrosis (Rosenfeld et al. (1992); Rich et al. (1993)) and α₁-antitrypsin deficiency (Lemarchand et al. (1992)). Although other alternatives for gene delivery, such as cationic liposome/DNA complexes, are also currently being explored, none as yet appear as effective as adenovirus mediated gene delivery. Adenoviral vectors currently being tested for gene therapy applications typically are deleted for Ad2 or Ad5 DNA to render them replication incompetent.

Although adenoviral vectors offer several advantages over other modes of gene delivery vehicles, they still exhibit some characteristics which impose limitations to their efficient use in vivo. These limitations primarily result in the limited ability of the vectors to efficiently deliver and target therapeutic genes to the tumor deposits. Researchers have attempted to circumvent this problem by administering large quantities of the delivery agent into the tumor environment but this is unlikely to be feasible when treating a dispersed metastatic disease. Recently it has been proposed that a solution to this issue might lie in the use of viral vectors which would retain the ability to replicate in tumor tissue and thereby amplify the effect of any therapeutic gene carried by the virus (S. J. Russell., 1994, European Journal of Cancer 8, 1165-1171). The potential use of replicating viruses in the treatment of cancer has a long history (Id.) and a great many virus types have been used in experimental trials as cancer therapeutics with no significant success.

Thus, there exists a need for methods which specifically target the therapeutic gene to the abnormally proliferating cells and also allow high copy numbers of the therapeutic gene to achieve greater efficacy by enabling efficient penetration of the diseased tissue. The present invention satisfies this need and provides related advantages as well.

### SUMMARY OF THE INVENTION

This invention provides a method of treating cancer by administering a replication competent adenoviral vector comprising a therapeutic gene and a disease specific gene regulatory region operationally linked to at least one replication gene. The replication competent targeted adenoviral vector preferentially replicates in the tumor cells following activation of the tumor specific gene regulatory region thereby amplifying the effect of the therapeutics gene carried by the replication competent adenoviral vector. This invention enables for the first time the targeting of a therapeutic gene for treating cancer using small amounts of viral vectors which selectively replicate to deliver therapeutic dosages of the therapeutic gene.

### BRIEF DESCRIPTION OF THE DRAWINGS

FIG. 1. Schematic Representation of rAd/AFP-E1a/TK. Adenovirus type 5 sequences containing the E1a promoter between nucleotides 355 and 483 have been deleted and replaced with a 1.7 kb fragment containing the alpha-fetoprotein enhancer/promoter. In addition Adenovirus type 5 sequences in the E3 region between Ad5 coordinates 28583 and 30470 have been deleted and in their place is inserted a 1130 base pair fragment corresponding to the HSV-1 thymidine kinase gene.

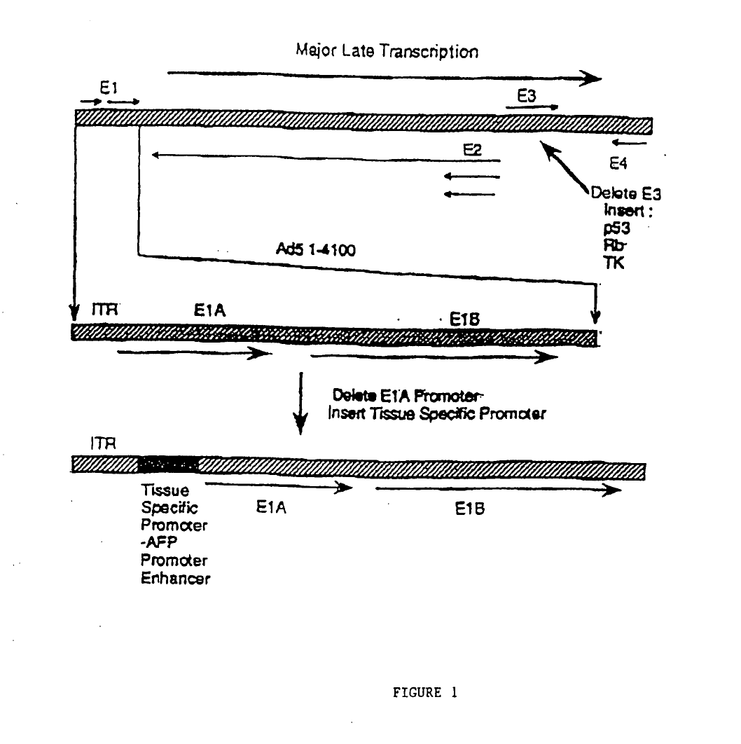

FIG. 2. Replication of rAd/AFP-E1a/TK in hepatocellular carcinoma (HCC) cell lines. rAd/AFP-E1a/TK and the replication competent control virus d1327 were used to infect two HCC cell lines. D1327 is deleted for the same region of E3 deleted in rAd/AFP-E1a/TK but contains the normal E1a promoter region. This Hep 3D cell line produces alpha-fetoprotein while the HLE cell line does not. Replication was assessed by isolating viral DNA and performing Southern blot analysis at the indicated timepoints. Replication, as assessed by radioactive probes, was measured using a Molecular Dynamics Phosphorimager. Results are normalized to the replication of the control virus d1327 in these cell lines. A) Replication of rAd/AFP-E1a/TK replication in Hep-3B cells. B) Replication of rAd/AFP-E1a/TK in HLE cells.

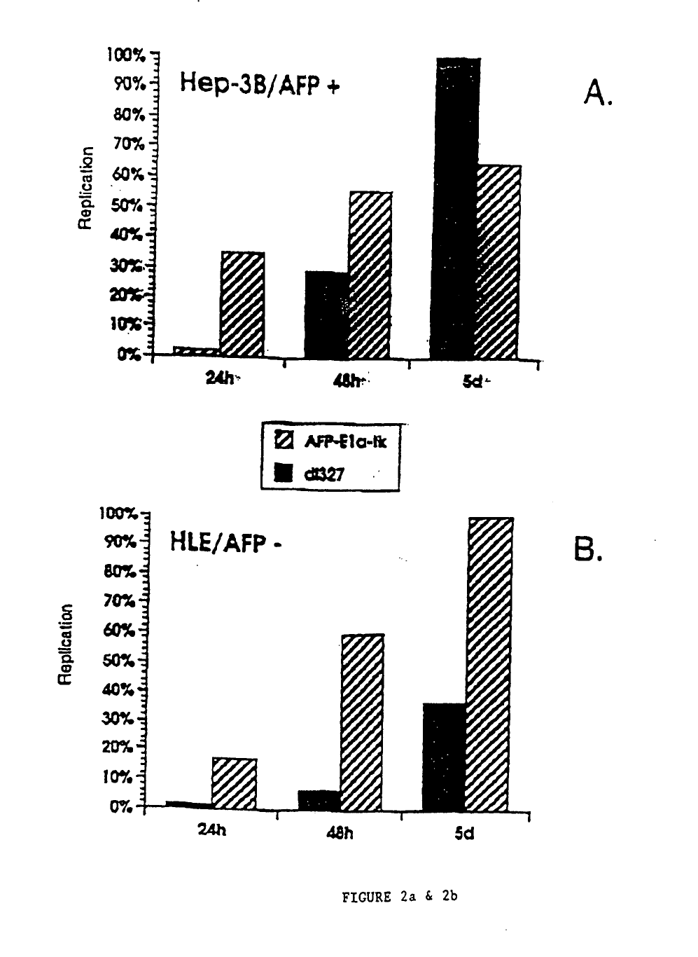

FIG. 3. Comparison of AD/AFP-E1a/TK replication in Hep-3B (AFP positive) vs HLE (AFP) negative cell lines normalized to replication of d1327 virus at each timepoint. rAd/AFP-E1a/TK replicates preferentially in AFP positive HCC cells.

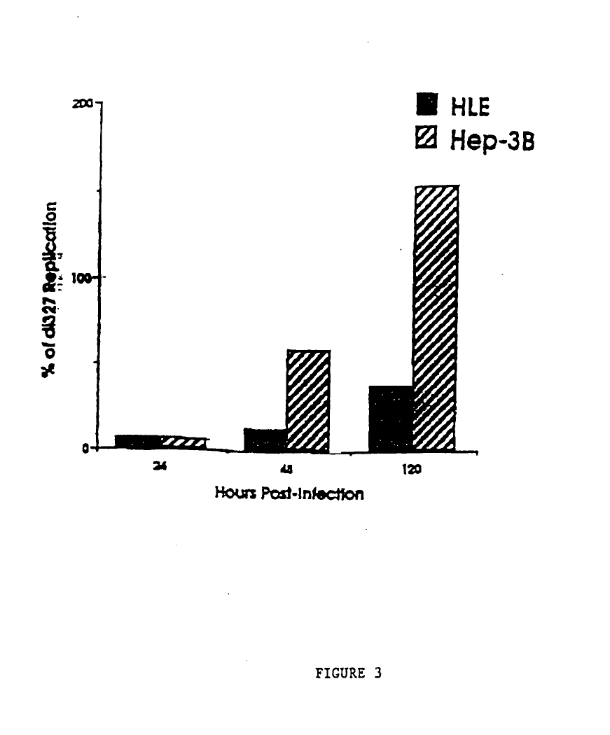

### DETAILED DESCRIPTION OF THE INVENTION

This invention is directed to gene therapy and to the use of disease specific replication competent adenoviral vectors for selectively expressing therapeutic genes at a particular site of interest, namely within a cancer cell. The use of replication competent vectors is advantageous in that therapeutic genes can initially be delivered to a small number of tumor cells where they are amplified by viral replication and able to be transferred to adjacent cells. Thus, the replication increases the overall efficiency of the gene delivery step and thus, increases the efficacy of the gene therapy protocol. The normal immune system of the host will prevent spread of virus throughout the body.

In one embodiment, the invention is directed to the therapeutic use of engineered replication competent recombinant adenoviruses to treat cancer and other hyperproliferative disorders or diseases in which there is a unique factor substance which would allow targeted delivery of a therapeutic substance using the method of this invention. The viruses have been modified to reduce their ability to replicate in normal cells while retaining their ability to replicate efficiently in specific tumor types. The adenoviral vectors include therapeutic genes such as cytotoxic genes or tumor suppressor genes which are lethal or otherwise render the cancer non-malignant or anti-sense compounds to certain viruses such as hepatitis or cytomegalovirus, or anti-viral compounds such as interferon-alpha. The tumor specific replication competent vectors have been engineered such that the promoter of the adenoviral E1a gene has been replaced with a tumor specific promoter/enhancer. An important distinction between these recombinant viruses and those typically used for gene therapy is that a replication gene such as the E1 gene, themselves are retained in the resulting recombinant adenoviruses. Because the viral E1 gene controls transcription of many other important viral genes (Horowitz, 1990) this modification restricts virus replication to those tumors which utilize the tumor specific promoter/enhancer inserted in place of the E1a promoter. One example of a cytotoxic gene is the Herpes simplex type-1 thymidine kinase gene which itself has a selective toxicity to replicating cells in the presence of the drug ganciclovir (F. L. Moolten, 1986). Replication of the recombinant adenovirus within the tumor mass amplifies the effect of the cytotoxic gene carried by the virus.

As used herein, the term “therapeutic gene” refers to a nucleic acid sequence which encodes a protein having a therapeutically beneficial effect such as regulating the cell cycle or inducing cell death. Examples of genes which regulate the cell cycle include p53, RB and mitosin whereas a gene which induces cell death includes the conditional suicide gene thymidine kinase. Cytokines which augment the immunological functions of effector cells are also included within the term as defined herein. Therapeutic genes are essentially foreign genes which are expressed from the replication competent adenoviral vectors used in the methods of the invention. These foreign genes are therefore DNA molecules which are not present in the exact orientation and position as the counterpart DNA molecule found in wild-type adenovirus. The foreign gene can be a DNA molecule up to about 4.5 kilobases.

The therapeutically beneficial effects of such genes can be conferred by either a direct or indirect 5 mode of action. For example, a therapeutic gene which acts directly can include those genes which are necessary for cell proliferation. Examples of such direct acting genes are the tumor suppressor genes and cell cycle regulatory genes. Examples of therapeutic genes which are beneficial through an indirect mode of action are genes which exhibit cytotoxic characteristics and immunomodulatory genes. Cytotoxic genes can be therapeutically beneficial either alone or when used in combination with other agents.

Included within the definition of the therapeutic genes of the invention are active fragments thereof and genes which contain minor modifications which do not significantly effect the intended function of the gene product. Thus, “active fragments” of therapeutic genes include smaller portions of the gene that retain the ability to encode proteins having therapeutic benefit. p56^{RB}, described more fully below, is but one example of an active fragment of a therapeutic gene which is a tumor suppressor gene. Modifications of therapeutic genes which are contemplated include nucleotide additions, deletions or substitutions, so long as the functional activity of the unmodified gene is retained. Thus, such modifications result in equivalent gene products that depart from the linear sequence of the naturally occurring proteins or polypeptides, but which have amino acid substitutions that do not change its biological activity. These equivalents can differ from the native sequences by the replacement of one or more amino acids with related amino acids, for example, similarly charged amino acids, or the substitution or modification of side chains or functional groups.

As used herein, the term “operationally linking” refers to the joining of an encoding nucleic acid sequence to expression elements which results in the biological production of the desired polypeptide. Therefore, “expression elements,” as used herein, refers to all nucleic acid elements which direct the proper transcription, processing, translation and sorting of a gene product from an encoding nucleic acid. Such elements can include, for example, promoters and regulatory elements such as the tumor specific promoter/enhancer as described herein, splicing sequences, translation initiation and termination sequences and signal sequences.

As used herein, the term “replication competent adenoviral vector” or “adenoviral vector” refers to vectors derived from the adenoviral genome which preferentially replicate in cancer cells and thus amplify the effect of the therapeutic gene carried by the virus. The replication of the vector is dependent on the presence of a factor(s) characteristic of the diseased tissue. The factor(s) trigger replication of the vector and in turn amplification of the therapeutic effect. The adenoviral vectors of this invention are engineered as described herein to reduce or eliminate their ability to replicate in normal cells while retaining their ability to replicate efficiently in specific tumor disease cell types.

As used herein, the term “tumor specific gene regulatory region” or “tumor specific regulatory region” or “tumor specific promoter” or “tumor specific promoter/enhancer” refers to transcription and/or translation regulatory regions that function selectively or preferentially in a specific tumor cell type. Selective or preferential function confers specificity to the gene therapy treatment since the therapeutic gene will be primarily expressed in a targeted or specific tumor cell type. Tumor specific regulatory regions include transcriptional, mRNA maturation signals and translational regulatory regions that are tumor cell type specific. Transcriptional regulatory regions include, for example, promoters, enhancers and silencers. Specific examples of such transcriptional regulatory regions include the promoter/enhancer elements for alpha-fetoprotein, carcinoembryonic antigen and prostate specific antigen. RNA processing signals include, for example, tissue specific intron splicing signals whereas translational regulatory signals can include, for example, mRNA stability signals and translation inition signals. Thus, tumor specific regulatory regions include all elements that are essential for the production of a mature gene product in a specific tumor cell type.

As used herein, the term “tumor suppressor gene” refers to a gene that encodes a protein that effectively inhibits a cell from behaving as a tumor cell. A specific example of a tumor suppressor gene is the retinoblastoma (RB) gene. The complete RB cDNA nucleotide sequences and predicted amino acid sequences of the resulting RB protein (designated p110^{RB}R) are shown in Lee et al. (1987). A truncated version of p110^{RB}, called p56^{RB} also functions as a tumor suppressor gene and is therefore useful as a therapeutic gene. The sequence of p56^{RB} is described by Huang et al. (1991). Tumor suppressor genes other than RB include, for example, the p16 protein (Kamb et al. (1994)), p21 protein, Wilm's tumor WT1 protein, or colon carcinoma DCC protein or related molecules such as mitosin and H-NUC. Mitosin is described in Zhu and Lee, U.S. application Ser. No. 08/141,239, filed Oct. 22, 1993, and a subsequent continuation-in-part by the same inventors, attorney docket number P-CJ 1191, filed Oct. 24, 1994, both of which are herein incorporated by reference. Similarly, H-NUC is described by W-H Lee and P-L Chen, U.S. application Ser. No. 08/170,586, filed Dec. 20, 1993, herein incorporated by reference.

Also encompassed within the definition of a tumor suppressor protein is any protein whose presence suppresses the neoplastic phenotype by reducing or eliminating the tumorigenicity, malignancy or hyperproliferative phenotype of the host cell. The neoplastic phenotype is characterized by altered morphology, faster growth rate, higher saturation density, growth in soft agar and tumorigenicity. The therapeutic genes described above encode proteins which exhibit this activity. “Tumorigenicity” is intended to mean having the ability to form tumors or capable of causing tumor formation and is synonymous with neoplastic growth. “Malignancy” is intended to describe a tumorigenic cell having the ability to metastasize and endanger the life of the host organism.

“Hyperproliferative phenotype” is intended to describe a cell growing and dividing at a rate beyond the normal limitations of growth for that cell type. “Neoplastic” also is intended to include cells lacking endogenous functional tumor suppressor protein or the inability of the cell to express endogenous nucleic acid encoding a functional tumor suppressor protein.

As used herein, the term “cell cycle regulatory gene” refers to genes encoding proteins which directly or indirectly control one or more regulatory steps within the cell cycle. Such cell cycle regulatory steps include, for example, the control of quiescent to proliferative phenotypes such as the G₀ G₁ transition as well as progression into apoptosis. Examples of cell cycle regulatory genes include the cyclins and cyclin dependent kinases.

As used herein, the term “immunomodulatory gene” refers to genes encoding proteins which either directly or indirectly have an effect on the immune system which augments the host's inherent response toward proliferating tumor cells. Such immunomodulatory genes include, for example, cytokines such as interleukins and interferons which are recognized by effector cells of the immune system.

As used herein, the term “cytotoxic gene” refers to a gene that encodes a protein which either alone or in combination with other agents is lethal to cell viability. Examples of cytotoxic genes which alone are lethal include toxins such as pertussis toxin, diphtheria toxin and the like. Examples of cytotoxic genes which are used in combination with other agents to achieve cell lethality include, for example, herpes simplex-1 thymidine kinase and cytosine deaminase. The subject is then administered an effective amount of a therapeutic agent, which in the presence of the anti-tumor gene is toxic to the cell. In the specific case of thymidine kinase, the therapeutic agent is a thymidine kinase substrate such as ganciclovir (GCV), 6-methoxypurine arabinonucleoside (araM), or a functional equivalent thereof. Both the thymidine kinase gene and the thymidine kinase metabolite must be used concurrently to be toxic to the host cell. However, in its presence, GCV is phosphorylated and becomes a potent inhibitor of DNA synthesis whereas araM gets converted to the cytotoxic anabolite araATP. Other anti-tumor genes can be used as well in combination with the corresponding therapeutic agent to reduce the proliferation of tumor cells. Such other gene and therapeutic agent combinations are known by one skilled in the art. Another example would be the vector of this invention expressing the enzyme cytosine deaminase. Such vector would be used in conjunction with administration of the drug 5-fluorouracil (Austin and Huber, 1993), or the recently described E. Coli Deo A gene in combination with 6-methyl-purine-2′-deosribonucleoside (Sorscher et al., 1994).

The invention provides a method of treating mammalian cancer cells. The method consists of administering a replication competent targeted adenoviral vector comprising a therapeutic gene and a disease specific gene regulatory region operationally linked to at least one replication gene wherein the disease cells activate the disease specific gene regulatory region.

Contrary to what has been known in the art, this invention claims the use of replication competent recombinant adenoviruses which selectively replicate at a selected site. Following infection the viral genome localizes to the cell's nucleus. Adenoviral replication then proceeds by initial transcription of the E1a gene. The products of the E1a gene then activate transcription of the other early transcription units, E1b, E2, E3 and E4. These products initiate DNA synthesis at which point the major late transcription unit is activated leading to synthesis of the major viral structural proteins and virus assembly in the nucleus.

The replication competent vectors of the invention are disease specific in that they replicate preferentially in the targeted tumor cell type. This tumor specific replication competence is achieved by operationally linking at least one gene for replication to a tumor specific gene regulatory region. Genes necessary for replication are any of those described above such as the E1a gene. Although other genes such as E2, E4 and the major late transcription unit can achieve tumor specific replication competence, the use of E1a is advantageous in that it also controls the expression of other adenoviral genes necessary for propagation. Thus, the invention provides for adenoviral vectors which retain the E1 genes and those which retain the E1a gene.

The replication competent adenoviral vectors useful in the methods of this invention can be modified so as to achieve a desired function for a particular need. Such modifications include additions, deletions or substitutions of adenoviral or exogenous sequences so as to augment the delivery and efficacy of the therapeutic gene. Further, adenoviral vectors based on any group C virus, serotype 1, 2, 5 and 6, can be used in the methods of this invention as well as vectors such as an Ad2/Ad5 based adenoviral vector.

The invention provides for therapeutic genes which are cytotoxic genes such as the conditionally lethal herpes simplex thymidine kinase gene. The invention also provides for therapeutic genes which are tumor suppressor genes. Examples of tumor suppressor genes include, for example, p53, RB, RB mutants, p21, p53 mutants or mitosin. Expression of such a therapeutic gene results in the restoration of the control of the cell cycle progression. The therapeutic genes can be under the control of a inducible promoter so that preferential tissue specific expression relies on the tumor specific expression of an essential replication gene. Alternatively, the therapeutic genes can similarly be under the control of a tumor specific gene regulatory region. The combined tumor specific expression of both a replication gene and the therapeutic gene is advantageous in that greater specificity is achieved and therefore greater efficacy of the methods are obtained.

Therapeutic genes which are cytotoxic can be directly lethal to achieve cell death of the targeted tumor cells or they can be, for example, conditionally lethal such as suicide genes which are used in conjunction with an agent which is capable of becoming toxic when metabolized by the suicide gene. A specific example of such a suicide gene is the herpes simplex thymidine kinase (TK) gene.

Expression cassettes can be incorporated into the replication competent vectors of the invention to allow greater flexibility to modify the vectors with a variety of genes necessary for a particular application. An expression cassette is therefore a functional term to describe the ability of the vector to achieve the recombinant production of the therapeutic gene of interest.

The invention provides tumor specific replication competent vectors wherein the gene regulatory regions are selected from the group consisting of the alpha-fetoprotein promoter/enhancer, the carcinoembryonic antigen promoter/enhancer, the tyrosinase promoter/enhancer and the prostate specific antigen promoter/enhancer. For other diseases such as inflammatory conditions, the inducer could be TNF-α and the responding regulatory element the interleukin-6 (IL-6) promoter. The therapeutic gene can encode interleukin-10 (IL-10) or another anti-inflammatory cytokine.

The vectors useful in the methods of this invention replicate specifically in specific tumor cells. The tumor specificity results from the incorporation of tumor specific gene regulatory regions which drive the expression of one or more genes which are essential for replication. Such elements include, for example, the alpha-fetoprotein promoter/enhancer, the carcinoembryonic antigen promoter/enhancer, the tyrosine promoter/enhancer and the prostate specific antigen promoter/enhancer. Each of these gene regulatory regions functions preferentially in specific tumor cell types. For example, the alpha-fetoprotein promoter/enhancer functions preferentially in hepatocellular carcinoma tumor cells. The carcinoembryonic antigen promoter/enhancer functions preferentially in colon cancer and breast tumor cells whereas the prostate specific antigen promoter/enhancer functions in prostrate tumor cells. Finally, the tyrosine promoter enhancer preferentially functions in melanoma tumor cells. Thus, the invention provides for the treatment of cancers including, for example, breast cancer, colorectal cancer, hepatocellular carcinoma and melanoma cancer.

Administration of the replication competent vectors is accomplished by methods well known to those skilled in the art. Such administration can be either alone or in acceptable pharmaceutical mediums.

A pharmaceutically acceptable carrier can contain a physiologically acceptable compound that acts, for example, to stabilize the composition or to increase or decrease the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the polypeptide and on the particular physio-chemical characteristics of the specific polypeptide. For example, a physiologically acceptable compound such as aluminum monosterate or gelatin is particularly useful as a delaying agent, which prolongs the rate of absorption of a pharmaceutical composition administered to a subject. Further examples of carriers, stabilizers or adjuvants can be found in Martin, Remington's Pharm. Sci., 15th Ed. (Mack Publ. Co., Easton, 1975), incorporated herein by reference. The pharmaceutical composition also can be incorporated, if desired, into liposomes, microspheres or other polymer matrices (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

The replication competent vectors can be administered as pharmaceutical compositions which include the vectors described herein in combination with one or more of the above pharmaceutically acceptable carriers. The compositions can then be administered therapeutically or prophylactically. Methods of administering a pharmaceutical containing the vector of this invention, are well known in the art and include but are not limited to, administration orally, intra-tumorally, intravenously, intramuscularly or intraperitoneal. Administration can be effected continuously or intermittently and will vary with the subject and the condition to be treated, e.g., as is the case with other therapeutic compositions (Landmann et al. (1992); Aulitzky et al. (1991); Lantz et al. (1990); Supersaxo et al. (1988); Demetri et al. (1989); and LeMaistre et al. (1991)).

It is understood that modifications which do not substantially affect the activity of the various embodiments of this invention are also included within the definition of the invention provided herein. Accordingly, the following examples are intended to illustrate but not limit the present invention.

### EXAMPLE I

A recombinant adenovirus vector has been constructed which is distinct from wild-type Adenovirus type 5 in two ways. First, the E1a promoter contained between Ad5 coordinates 355 and 483 has been deleted and replaced with a 1.7 kb fragment encoding the alpha-fetoprotein (AFP) enhancer/promoter. Second, the E3 region between AdS coordinates 28583 and 30470 has been deleted and in its place we have inserted the HSV-1 TK gene. The DNA deleted in E3 is non-essential for virus replication. The recombinant virus vector, by virtue of its AFP control elements replicates preferentially in AFP cancer cells. This allows the effect of the therapeutic gene contained in E3, in this example HSV-1 TK, to be amplified preferentially in those cells in which the tumor specific promoter is activated. The AFP promoter is activated in hepatocellular carcinomas as well as other cancers and this recombinant replication competent adenovirus vector provides a means of treating these cancers. Together tumor specific promoters can be inserted in place of the AFP enhancer/promoter in order to amplify the virus in other tumor types. This virus can be administered either systemically or by intratumoral injection. Because it is self replicating only a small amount of virus is required to initiate therapy of the tumor cells.

Methods—All plasmid and viral constructs were constructed using standard methods (Sambrook et al. 1989, Graham and Prevec, 1991).

Recombinant Plasmid Constructions—E1 region. To place the E1a gene under control of a tumor specific promoter plasmids were constructed using standard methods. Plasmid pcDNA3 Ad2 E1 was constructed by cloning the adenovirus type 2 E1a gene into the commercially available vector pcDNA3 (Invitrogen Corp.). The E1a gene was isolated by polymerase chain reaction upon pure Ad2 DNA (Gibco/BRL) using the following primers:

| | |
|---|---|
| 5′ Ela PCR primer: | CTG AAG CTT GAG TTC CTC AAG AGG |
| | CCA CTC |
| 3′ Ela PCR primer: | GCG CTC GAG ATT TAA CAC GCC ATG |
| | CAA GTT |

The 1189 bp E1a PCR product was run on a 1% agarose gel and the band was excised via razor blade and purified from the agar via Geneclean II (Bio101 Inc.) The purified PCR product was then digested with Xhol and Hind III and cloned into the Hind III and Xho 1 site of pcDNA 3 to generate pcDNA3 Ad2 E1a. Plasmids pAd/AFP/B was constructed by cloning the alpha fetoprotein enhancer/promoter between the X and Y sites of the adenovirus transfer vector plTR B. This vector was constructed similarly to pAANTK which is described below. To construct pAd/AFP/E1a the Ad2 E1a gene was isolated from pcDNA3 as an HindIII (blunted with Klenow polymerase)/Ncol restriction fragment and inserted adjacent to the AFP promoter in pAd/AFB/B between the Xbal (blunted with Klenow polymerase) and Ncol sites.

Recombinant Plasmid Constructions—E3 region. In order to insert a therapeutic gene such as a cytotoxic gene into the adenoviral E3 region we constructed the plasmid pSE280-E3 delta as described below. The construct was generated by cloning an AdS restriction fragment (Munl/Dral) corresponding to AdS nucleotide coordinates 26045 to 38711 between the EcoRI and Smal sites of pSE280 (Invitrogen Corp.). The resulting plasmid pSE280 E3 5′ was then cut with restriction enzymes Nhel and SnaBl and a second restriction fragment of adenoviral DNA (Xbal/EcoRV) corresponding to Ad5 nucleotides 30471-33756 was inserted. The resulting plasmid pSE280-E3 delta contains the adenoviral E3 region except for a deletion corresponding to adenoviral coordinates 28711-30471. These sequences are not essential for adenoviral replication and foreign genes can be inserted into the region. To insert the TK gene into this region a TK gene fragment isolated by PCR of the TK gene and flanked by Xbal and BamHl restriction sites was isolated by polymerase chain reaction upon pAANTK and cloned into the Xbal and BamHl sites of pSE280-E3 delta to generate pSE280/E3 delata/TK.

The plasmid pACNTK which is similar to pAANTK was constructed by subcloning the HSV-TK gene from pMLBKTK (ATCC No. 39369) into the polylinker of a cloning vector, followed by isolation of the TK gene with the desired ends for cloning into the pACN vector. The pACN vector contains adenoviral sequences necessary for in vivo recombination to occur to form recombinant adenovirus. The construction of the plasmid pAANTK entailed the PCR amplification of fragments encoding the α-fetoprotein enhancer (AFP-E) and promoter (AFP-P) regions subcloned through several steps into a final plasmid where the AFP enhancer and promoter are upstream of the HSV-TK gene followed by adenovirus Type 2 sequences necessary for in vivo recombination to occur to form recombinant adenovirus.

Construction of Recombinant Adenoviruses—To generate a recombinant adenovirus in which the E1a promoter has been replaced by a tumor specific promoter, the plasmid pAd/AFP/E1a was cut with the restriction enzyme Clal and then ligated to the adenovirus d1309 (Jones and Shenk, 1979) which had also been cut with Clal. This ligated DNA was used to transfect 293 cells and the resulting virus plaques were screened by restriction analysis for the insertion of the AFP promoter. The resulting virus was called rAd—AFP—E1a/309. The HSV-1 TK gene was then replaced into the E3 region of Ad-AFP-E1a/309 by cutting the DNA of this virus with the restriction enzymes EcoRl and Srfl and then co-transfecting the viral DNA into 293 cells with pSE280-E3 delta/TK DNA cut with BstE1l and Kpnl. Recombinant viral plaques resulting from in vivo recombination were isolated and screened by restriction analysis for the presence of the TK gene inserted into the E3 region.

Analysis of Viral Replication

1×10E+6 of Hep3B (AFP positive HCC cell line) and HLE (AFP negative cell line) cells were seeded in 10 cm tissue culture plates. After 24 hours the cells were infected with rAd/AFP-E1a-TK or d1327 at MOI 1. Viral DNA was harvested from the infected cells after 24 hr, 48 hr, and 5 day post-infection. The viral DNA was prepared for analysis as follows:
1. Remove the cell medium and wash once with the HBSS buffer.
2. Trypsinize the cells with 1× trypsin-EDTA.
3. Pellet the cells in the Beckman TJ-6 table top centrifuge at speed 6 for 5 min.
4. Resuspend the cell pellet with ice-cold PBS twice.
5. Add 650 ul of Hirt lysis buffer: 10 mM Tris (pH7.5), 10 mM EDTA, 0.6% SDS, and 163 ul of 5M NaCl to each cell pellet. Incubate at −20° C. for 1 hr.
6. Spin the samples at room temperature in the microfuge for 30 min. Transfer the supernatant into microcentrifuge tube.
7. Add proteinase K to 200 ug/ml and incubate tubes at 37° C. for 1 hr.
8. Extract the viral DNA with an equal volume of phenol:chloroform/isoamyl alcohol (49:1) once and then with equal volume of chloroform once.
9. Precipitate the viral DNA with 2 volume of 100% ethanol and wash the pellet with 70% of ethanol.
10. Resuspend the pellet in 29 microliters of TE pH 8.0.

10 microliters of each viral DNA sample was digested with restriction endonuclease Xho 1 at 37° C. overnight. The digested DNA samples were run on a 0.8% agarose gel at 20 v overnight. The digested DNA was transferred from the gel to a nylon membrane using a Stratagene Posiblot pressure blotter. To detect adenoviral replication the membrane was probed with a 32-P probe which contains sequence corresponding to 1711-2266 of Ad2. The blot was exposed to a phosphoimager screen for 1 hour and the autoradiographic image was acquired and quantitated using a Molecular Dynamics phosphorimager. Replication data for each cell line was compared to replication of the wild-type virus d1327 in that cell line.

Results

To assess the replication potential of rAd/AFP-E1a-TK the virus was used to infect cell lines which either utilize the AFP promoter (Hep-3B) or do not utilize this promoter (HLE). After the initial infection at a multiplicity of infection of 1, viral DNA was harvested at 1, 2 or 5 days and analyzed by Southern blot analysis and quantitated using a Molecular Dynamics phoshorimager. As a control and standard the cells were also infected with the replication competent adenovirus d1327. D1327 is a wild-type adenovirus from which the same non-essential segment of E3 has been deleted which is deleted in rAd/AFP-E1a-TK and therefore serves as an appropriate control of viral replication. By comparing the replication of rAd/AFP-E1a-TK to that of d1327 in each of the two cell lines it is possible to assess the effect of replacing the viral E1a promoter with the AFP promoter/enhancer. These experiments indicated that this replacement placed the rAd/AFP-E1a-TK at a replicative disadvantage compared to d1327 in the AFP negative HLE cell line. In contrast rAd/AFP-E1a-TK replicated much more efficiently in the AFP positive tumor cell line. Although the regulation is not absolute, there is a 4 to 5 fold replication advantage in the AFP positive versus negative cell line.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1-18. (canceled)

19. A replication competent adenoviral vector comprising a tumor specific transcription regulatory sequences operably linked to at least one replication gene of the adenoviral vector.

20. The replication competent adenoviral vector of claim 19, wherein the tumor specific transcription regulatory sequences is an α-fetoprotein promoter/enhancer.

21. The replication competent adenoviral vector of claim 19, wherein the tumor specific transcription regulatory sequences is a carcinoembryonic promoter/enhancer.

22. The replication competent adenoviral vector of claim 19, wherein the tumor cells are selected from the group consisting of hepatoceullar carcinoma cells, breast cancer cells, colorectal cancer cells and melanoma cells.

23. The replication competent adenoviral vector of claim 19, wherein the tumor specific transcription regulatory sequences is a tyrosinase promoter/enhancer.

24. The replication competent adenoviral vector of claim 19, wherein vector further comprises a DNA sequence of interest.

25. The replication competent adenoviral vector of claim 24, wherein the DNA sequence is a cytotoxic gene.

26. The replication competent adenoviral vector of claim 24, wherein the DNA sequence is a tumor suppressor gene.

27. The replication competent adenoviral vector of claim 26, wherein the tumor suppressor gene is selected from the group consisting of p53, p21, p53 mutants, p16, Rb and Wilm's tumor WT1 protein.

28. The replication competent adenoviral vector of claim 19, wherein the replication gene is an E1 gene.

29. The replication competent adenoviral vector of claim 28, wherein the replication gene is the E1a gene.

30. The replication competent adenoviral vector of claim 19, wherein the replication gene is an E2 gene.

31. The replication competent adenoviral vector of claim 19, wherein the replication gene is an E4 gene.

32. The replication competent adenoviral vector of claim 25, wherein the cytotoxic gene is a thymine kinase gene.

33. The replication competent adenoviral vector of claim 24, wherein the DNA sequence of interest is an immunomodulator gene.

34. The replication competent adenoviral vector of claim 33, wherein the immunomodulator gene encodes a cytokine.

35. The replication competent adenoviral vector of claim 25, wherein the cytotoxic gene is a cytosine deaminase gene.

36. The replication competent adenoviral vector of claim 27, wherein the tumor suppressor gene is a p53 gene.

37. The replication competent adenoviral vector of claim 29, wherein the endogenous E1a promoter is replaced by the tumor-specific transcription regulatory sequence.

38. The replication competent adenoviral vector of claim 37, wherein the tumor-specific transcription regulatory sequence comprises an α-fetoprotein promoter.

39. The replication competent adenoviral vector of claim 38, wherein the vector further comprises an interferon gene.

40. The replication competent adenoviral vector of claim 39, wherein the interferon gene is an α-interferon-2B gene.

41. The replication competent adenoviral vector of claim 37, wherein the vector further comprises a p53 gene.

42. The replication competent adenoviral vector of claim 29, wherein the endogenous E1a promoter is replaced by the tumor-specific transcription regulatory sequence.

43. The replication competent adenoviral vector of claim 42, wherein the tumor-specific transcription regulatory sequence comprises an α-fetoprotein promoter.

44. The replication competent adenoviral vector of claim 43, wherein the vector further comprises an α-interferon-2B gene.

45. The replication competent adenoviral vector of claim 42, wherein the vector further comprises a p53 gene.

46. The replication competent adenoviral vector of claim 34, wherein the cytokine is an interferon.

47. The replication competent adenoviral vector of claim 46, wherein the interferon is α-interferon 2B.

48. The replication competent adenoviral vector of claim 46, wherein the cytokine is selected from the group consisting of IL-2, IL-4, IL-10 and IL-13.

49. The replication competent adenoviral vector of claim 21, wherein the vector further comprises a tumor suppressor gene.

50. The replication competent adenoviral vector of claim 49, wherein the tumor suppressor gene is p53.

51. The replication competent adenoviral vector of claim 49, wherein the endogenous E1 promoter is replaced by the carcinoembryonic antigen promoter/enhancer.
